(19) United States
(12) Patent Application Publication
(10) Pub. No.: US 2007/0191869 A1
(43) Pub. Date: Aug. 16, 2007

(54) LINEAR LIGATION BAND

(75) Inventors: Scott Wadsworth, St. Ignatius, MT (US); Jake Wadsworth, St. Ignatius, MT (US)

Correspondence Address: DYKAS, SHAVER & NIPPER, LLP, P.O. BOX 877, BOISE, ID, 83701-0877, US

(73) Assignee: Wadsworth Mfg. Inc., St. Ignatius, MT (US)

(21) Appl. No.: 11/619,109

(22) Filed: Jan. 2, 2007

Related U.S. Application Data
(60) Provisional application No. 60/773,928, filed on Feb. 15, 2006.

(51) Int. Cl. A61B 17/08; A61B 17/10

(52) U.S. Cl. 606/151; 606/140

(57) ABSTRACT

A ligation band made up of a linear length of elastomeric material having first and second ends with connection portions adapted to connect with a tensioning mechanism portion of a ligating band application device. When in place upon a ligating band application device, the two ends are connected to the tensioning portion of the ligating band application device and the loop formed by the interconnection of these two ends upon the application device is tensioned by the tensioning member. The loop, once tensioned, can be held in a desired tensioned state around the body part that is to be removed by placing a grommet, clip or other form of holding device around the tensioned loop.

### CROSS-REFERENCE TO RELATED APPLICATIONS

This application claims the priority date of the provisional application entitled LINEAR LIGATION BAND filed by Wadsworth et al. on Feb. 15, 2006 with application Ser. No. 60/773,928.

### FIELD OF THE INVENTION

This invention generally relates to devices for ligating body parts such as ligation-type castrating tools and more particularly, to a system and device for removing body parts utilizing a ligation technique.

### BACKGROUND OF THE INVENTION

In modern animal husbandry it has become increasingly common, responsive to market demands, to castrate bovine bulls after their maturity rather than during infancy as heretofore has been common. Such castration by ligation has become popular because of the simplicity of the process and the benefits it provides in avoiding undesirable consequences such as microbial infection, insect invasion, excessive bleeding and the like. Ligation type castration of younger animals has been and is accomplished largely by use of small preformed continuous elastic or elastomeric bands. These small elastic bands are generally placed by a spreading type tool having two elongated arms pivotally interconnected in their medial portions to allow expansion of a band carried at one end portion of the tool against its elastic bias for placement over the scrotal pouch of the animal with subsequent release. This process works well with juvenile animals that do not have a mature testicular structure of larger size or with body parts that can easily fit within the opening defined by the loop, but it has not been viable with larger animals having mature testicular structure such as bovine bulls. The typical reason for this failure is that a small preformed elastic band that would contract to a small enough configuration to provide adequate compression so as to allow for ligation to take place usually cannot be expanded sufficiently so as to allow placement of the loop over the largest portion of the body part that is to be removed. This may be due to the inherent limitations which occur within the band or because of the physical limitations related to the application device that is being used to apply the device.

This problem has been recognized in the past and a solution presented by the instant inventor's U.S. Pat. No. 4,691,704 issued Sep. 8, 1987 and U.S. Pat. No. 5,188,637 issued Feb. 23, 1993, whereunder a length of elastic surgical tubing connected to a bulk of such material is formed in place within a tool, tensioned about an animal's scrotal pouch and fastened in its tensed condition by a clip or grommet. Though this process provides effective ligation castration of larger mature animals and has been accepted about the world, problems still remain with the process. It is an object therefore of the present invention to provide a method and device for removing body parts from animals which can be easily accomplished by unskilled workmen.

### SUMMARY OF THE INVENTION

The present invention is a ligation band for use in removing body parts through a ligation process. The band is made up of a linear length of elastomeric material having a first end and a second end. The first end and the second ends each having connection portions adapted to connect with a tensioning mechanism portion of a ligating band application device. When in place upon a ligating band application device, the two ends are connected to the tensioning portion of the ligating band application device and the loop formed by the interconnection of these two ends upon the application device is tensioned by the tensioning member. When this loop has been adequately and appropriately tensioned so as to provide a desired level of tension around the body part which is to be removed, this loop can be held in a desired tensioned state by placing a grommet, clip or other form of holding device around the tensioned loop. Once this has taken place, the elastomeric material between the ends of the band and the grommet can be cut and the remaining ends of the elastomeric material trimmed so as to be generally flush with the grommet or clip.

The connection portions of the present invention may be varied according to the particular necessity of the user, however it has been determined that hooks or loops which are configured to interact with a particular tensioning device are typically the most user friendly and convenient for use. Preferably these hooks or loops are made of a generally non-stretchable material such as wire or metal, however in some instances other generally non-stretchable material such as various types of durable plastic may also be utilized.

In one preferred embodiment of the invention, the connection portions of the first and second ends are wire loops that are wound around the length of elastomeric material in such a way so as to hold these loops in a close and non-slipping arrangement with the tubing.

In another embodiment of the invention, the ligation band is generally hollow and the connection portions that are connected to this ligation band are made up of a hook or loop that is connected to a post that also has a stopper. The post and the stopper are configured to be inserted within the hollow portion of the ligation band. A crimping device such as a grommet, clip, or other type of device is then applied to the outer surface of the ligation band and holds the post and the stopper in a desired position within the hollow portion of the ligation band. Preferably this clip or grommet is positioned at a location along the post between the stopper and the end of the device.

The part of the connection portion which is configured to attach to the tensioning member of the device is typically configured in the shape of a closed loop, however in some instances this loop may be opened, thus rendering a more hook-like type of shape.

The loop of the present invention may be utilized with various types of loop or band application devices so as to form a system for performing ligation castration. Most preferably, the linear length of material described above is utilized with an application device made up of an elongated tool body with a front end and a rear end. The front end of the elongated tool body having a portion adapted to hold a grommet through which the ends of the ligation band can be pulled by a tensioning member.

The loops of the present invention may be utilized with any of a variety of tensioning mechanisms including those devices that include a pulling rod, a winding spool, or other types of pulling devices. Regardless of the configuration of the particular tensioning device, the present invention can be pulled until a desired tension is achieved and then held in this desired position by a clip, grommet or other type of securing device.

In the preferred embodiment of the invention, the securing device is a grommet which is held within the front portion of the device and which is deformed around the tensioned band by deforming the grommet. This deformation of the grommet is preferably done by pivoting a lever which is connected to the tool body. This lever may be arranged in various ways including in line with the elongated body, generally perpendicular to the orientation of the tool body or in any other of a variety of different ways. In the preferred embodiment, a cutting means is then used to cut the band between the grommet or clip and the tensioning device.

This cutting means may be a knife, razor, scissors or other suitable cutting device. In some embodiments of the invention this cutting device is operatively connected to the application tool. In the preferred embodiment of the invention, the formation of loops and the placement of these loops around a body part that is to be ligated is assisted by a pair of arms that extend outward from the front portion of the elongated tool body and which allow placement and stretching of the device of the present invention over these arms.

In order for the bands of the present invention to have sufficient compressive force or bias so as to properly occlude the flow of blood and cause atrophy and removal of the undesired body part, various physical parameters have been found to be most beneficial. It has been found that a band of approximately one to two inches in diameter with a substantial cross-sectional area approximating 0.1 square inch in relaxed condition for natural rubber and varying somewhat, depending upon the elastic nature of other elastomeric material from which the band is formed is preferred. These parameters generally differ by several fold from the corresponding parameters of continuous elastic castration bands heretofore used for smaller immature animals or bands that are not stretched for placement and the parameters tend to dictate the nature of a tool used for their placement.

The prior art problem related to the inability of maintaining sufficient elastic tension to provide ligation by a continuous band that expands to create a large enough orifice to allow placement is solved by using a clip or grommet to hold the tensioned loop in a desired tensioned state around the body part which is to be removed.

The present invention provides a device and tool that provides a safe and effective process for allowing a single operator to accomplish a greater number of body part removal in a given period of time than could be accomplished with various prior art tools and devices.

The purpose of the foregoing Abstract is to enable the public, and especially the scientists, engineers, and practitioners in the art who are not familiar with patent or legal terms or phraseology, to determine quickly from a cursory inspection, the nature and essence of the technical disclosure of the application. The Abstract is neither intended to define the invention, which is measured by the claims, nor is it intended to be limiting as to the scope of the invention in any way.

Still other features and advantages of the present invention will become readily apparent to those skilled in this art from the following detailed description describing only the preferred embodiment of the invention, simply by way of illustration of the best mode contemplated by carrying out my invention. As will be realized, the invention is capable of modification in various obvious respects all without departing from the invention. Accordingly, the drawings and description of the preferred embodiment are to be regarded as illustrative in nature, and not as restrictive in nature.

### BRIEF DESCRIPTION OF THE DRAWINGS

FIG. 1 shows an embodiment of a preferred first embodiment of the ligation band of the present invention.

FIGS. 2 is a view of the preferred embodiment of the band of the present invention shown in FIG. 1 in use with an application device.

FIG. 3 is a detailed view of the interconnection of the band shown in FIG. 1 with an application device.

FIG. 4 is a detailed view of a preferred second embodiment of the present invention.

FIG. 5 is a detailed view of the connection device shown in FIG. 4.

FIG. 6 is a view of a third embodiment of the invention

FIG. 7 is a detailed view of the connection device shown in FIG. 8

FIG. 8 is a view of a fourth embodiment of the invention

### DESCRIPTION OF THE PREFERRED EMBODIMENTS

While the invention is susceptible of various modifications and alternative constructions, certain illustrated embodiments thereof have been shown in the drawings and will be described below in detail. It should be understood, however, that there is no intention to limit the invention to the specific form disclosed, but, on the contrary, the invention is to cover all modifications, alternative constructions, and equivalents falling within the spirit and scope of the invention as defined in the claims.

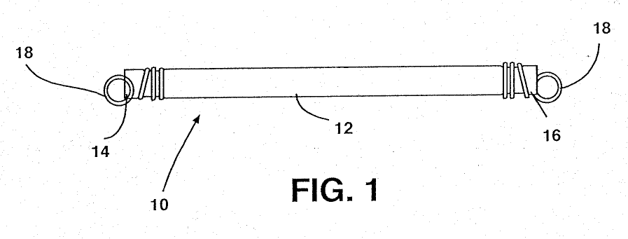

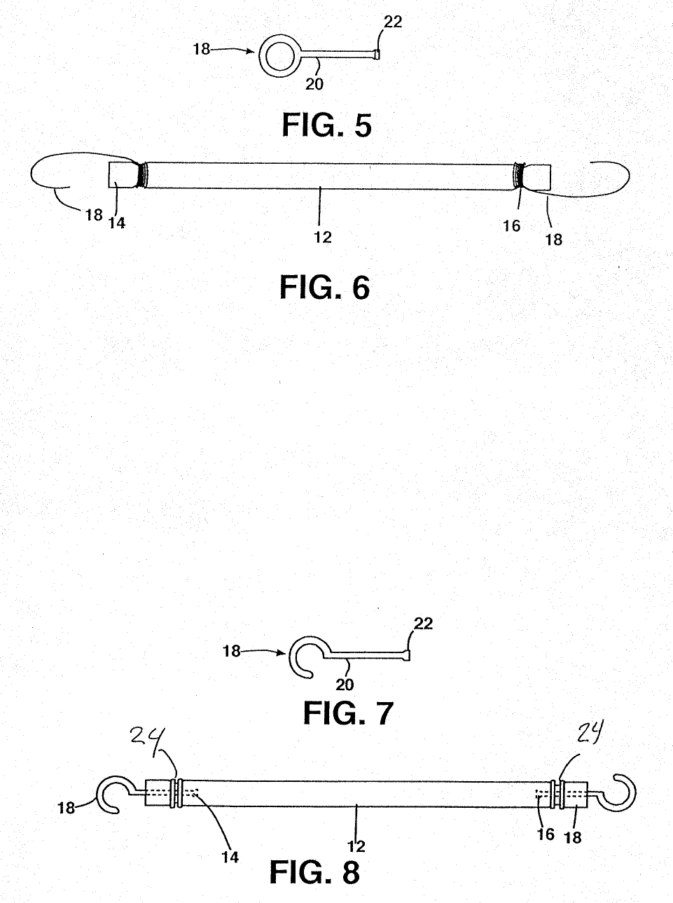

The present invention is a ligation band for use in removing body parts through a ligation process, and a system that includes a device that applies and tensions these bands. FIGS. 1-8 show the present invention in its outlined forms.

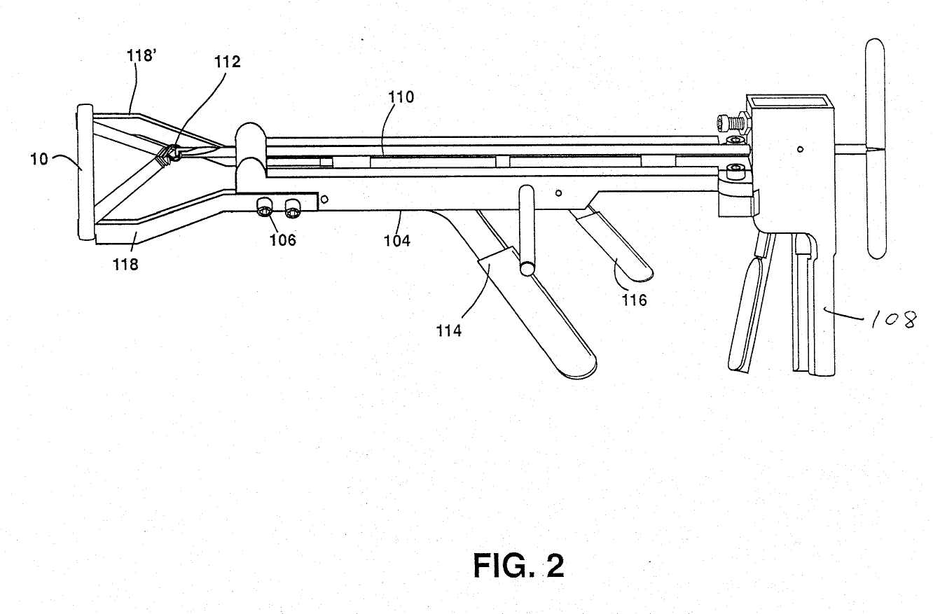

Referring first to FIG. 1, the band of the present invention is shown. This band 10 is made up of a linear length of material 12, having a first end 14 and extending along a length to a second end 16, attached at each of these ends are connection portions 18, 18′ which are configured to connect with a tensioning mechanism portion of a ligating band application device. An example of such a device with the band 10 of the present invention in place is shown in FIG. 2.

FIG. 2 shows an example of the application tool 102 with which the band of the present invention may be utilized. The application tool is made up of an elongated body 104 which has a front end 106 and a rear end 108. The front end 106 of the device includes a portion that is configured to hold a grommet therein. A tensioning mechanism 110 having a portion 112 configured to connect with the connection portions 18,18′ of the band 10 is connected to the elongated body. A pivoting lever 114 is also connected to the elongated body and is configured to be moved from a first position to a second position thereby crimping the grommet which is held in the front portion 106 of the device.

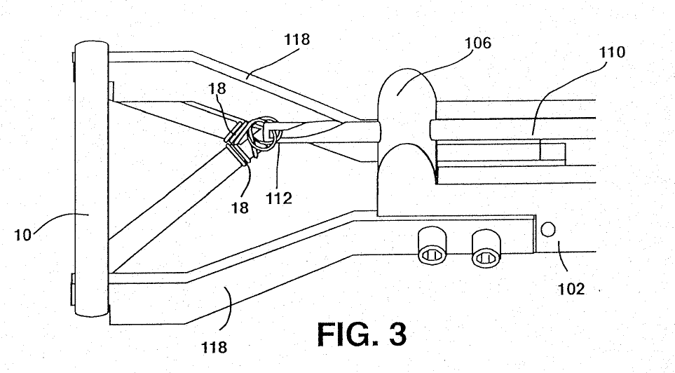

When in place upon a ligating band application device 102, the two ends 14, 16 of the band 10 are connected to a portion of the tensioning mechanism 112 and the loop formed by the interconnection of these two ends upon the application device is tensioned by the tensioning member 112. To assist in the formation of a ligation loop upon the tool a pair of arms 118,118′ are included upon the device. A detailed view of these arms and the loop are shown in FIG. 3.

When this loop has been placed around a body part that is to be removed and has been adequately tensioned so as to provide a desired level of tension around the body part which is to be removed, this loop can be held in a desired tensioned state around the body part by placing a grommet, clip or other form of holding device around the tensioned loop. Once this has taken place, the elastomeric material between the ends of the band and the grommet can be cut and the remaining ends of the elastomeric material trimmed so as to be generally flush with the grommet or clip.

In the preferred embodiment of the invention, the securing device is a grommet which is held within the front portion 106 of the application device 102 and which is deformed around the tensioned band by the lever 114. This lever may be arranged in various ways including in line with the elongated body, generally perpendicular to the orientation of the tool body or in any other of a variety of different ways. In the preferred embodiment, a cutting means 116 is then used to cut the band between the grommet or clip and the tensioning device.

This cutting means 116 may be a knife, razor, scissors or other suitable cutting device. In the preferred embodiment of the invention, this cutting device is operatively connected to the application tool.

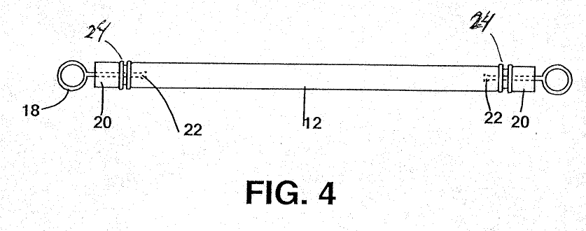

The bands 10 of the present invention contain connection portions 18, 18′ that may be varied according to the particular necessity of the user. However, it has been determined that hooks or loops which are configured to interact with a particular tensioning device are typically the most user friendly and convenient types of connection devices. Examples of various embodiments of these connection devices are shown in FIGS. 4-8. Preferably these hooks or loops which are utilized in the present invention are made of a generally non-stretchable material such as wire or metal. However, in some instances other generally non-stretchable material such as various types of durable plastic may also be utilized.

In one preferred embodiment of the invention shown in FIG. 1, the connection portions of the first and second ends are wire loops that are wound around the length of elastomeric material in such a way so as to hold these loops in a close and non-slipping arrangement with the tubing.

In another embodiment of the invention, shown in FIGS. 4, 5, 7 and 8 the length of ligation material 12 is generally hollow and the connection portions that are connected to this ligation band are made up of a hook or loop that is connected to a post 20 that is also connected to a stopper 22. The post 20 and the stopper 22 are configured to be inserted within the hollow portion of the ligation band 12. A crimping device 24 such as a grommet, clip, or other type of device is then applied to the outer surface of the ligation band and holds the post 20 and the stopper 22 in a desired position within the hollow portion of the ligation band. Preferably this clip or grommet is positioned at a location along the post 20 between the stopper 22 and the hook or loop portion.

While the part of the connection portion 18, 18′ which is configured to attach to the tensioning member of the device is typically configured in the shape of a closed loop, in some instances this loop may be opened, thus rendering a more hook-like type of shape.

While the ligation band 10 of the present invention is shown as being utilized with a particular type of application device, it may also be utilized with various other types of loop or band application devices so as to form a system for performing ligation castration. The linear ligation bands of the present invention may be utilized with any of a variety of tensioning mechanism including those devices that include a pulling rod, a winding spool, or other types of pulling devices. Regardless of the configuration of the particular tensioning device, the present invention can be pulled until a desired tension is achieved and then held in this desired position by a clip, grommet or other type of securing device.

In order for the bands of the present invention to have sufficient compressive force or bias so as to properly occlude the flow of blood and cause atrophy and removal of the undesired body part, various physical parameters have been found to be most beneficial. It has been found that a band of approximately one to two inches in diameter with a substantial cross-sectional area approximating 0.1 square inch in relaxed condition for natural rubber and varying somewhat, depending upon the elastic nature of other elastomeric material from which the band is formed is preferred. These parameters generally differ by several fold from the corresponding parameters of continuous elastic castration bands heretofore used for smaller immature animals or bands that are not stretched for placement and the parameters tend to dictate the nature of a tool used for their placement.

The present invention provides a device and tool that provides a safe and effective process for allowing a single operator to accomplish a greater number of body part removal in a given period of time than could be accomplished with various prior art tools and devices.

While there is shown and described the present preferred embodiment of the invention, it is to be distinctly understood that this invention is not limited thereto but may be variously embodied to practice within the scope of the following claims. From the foregoing description, it will be apparent that various changes may be made without departing from the spirit and scope of the invention as defined by the following claims.

What is claimed is:

1. A ligation band for ligating body parts comprising:
a linear length of elastomeric material having a first end and a second end, said first and second ends each having connection portions adapted to connect with a tensioning mechanism portion of a ligating band application device.

2. The ligation band of claim 1 wherein said connection portions of said first and second ends are loops.

3. The ligation band of claim 1 wherein said connection portions of said first and second ends are hooks.

4. The ligation band of claim 1 wherein said connection portions of said first and second ends are made from a non-stretching material.

5. The ligation band of claim 1 wherein said connection portions of said first and second ends are wire loops that are wound around said length of elastomeric material.

6. The ligation band of claim 1 wherein said linear length of material is generally hollow.

7. The ligation band of claim 6 wherein said connection portion of said first and second ends each comprise a post also having a stopper connected thereto, said post and said stopper configured for insertion into said hollow portion of said ligation band.

8. The ligation band of claim 7 further comprising at least one crimping device attached to said ligation material near said first and second ends.

9. The ligation band of claim 8 wherein said crimping device is located between said stopper and said end.

10. The ligation band of claim 9 wherein said connection portion is a loop.

11. The ligation band of claim 9 wherein said connection portion is a hook.

12. A system for ligating a body part comprising:
a linear length of elastomeric material having a first end and a second end, said first and second ends each having connection portions adapted to connect with a tensioning mechanism portion of a ligating band application device,
an application tool having an elongated tool body with a front end and a rear end, said front end of said elongated tool body having a portion adapted to hold a grommet therein;
a tensioning mechanism interconnected to said tool body, said tensioning mechanism having a portion configured to connect with said first and second ends of said linear length of ligation material, said tensioning mechanism configured to pull said first and second ends through said grommet toward said rear end of said elongated tool body;
a lever pivotally connected to said tool body for deforming said grommet around said elastomeric material; and
a cutting means adapted to cut said ligation material after said grommet has been deformed.

13. The ligation band of claim 12 wherein said connection portions of said first and second ends are loops.

14. The ligation band of claim 12 wherein said connection portions of said first and second ends are hooks.

15. The ligation band of claim 12 wherein said connection portions of said first and second ends are made from a non-stretching material.

16. The ligation band of claim 12 wherein said connection portions of said first and second ends are wire loops that are wound around said length of elastomeric material.

17. The ligation band of claim 12 wherein said linear length of material is generally hollow.

18. The ligation band of claim 17 wherein said connection portion of said first and second ends each comprise a post also having a stopper connected thereto, said post and said stopper configured for insertion into said hollow portion of said ligation band.

19. The ligation band of claim 18 further comprising at least one crimping device attached to said ligation material near said first and second ends.

20. The ligation band of claim 19 wherein said crimping device is located between said stopper and the end of said ligation tubing.

21. The ligation band of claim 20 wherein said connection portion is a loop.

22. The ligation band of claim 9 wherein said connection portion is a hook.

23. The system of claim 12 further comprising a pair of arms connected to said front end of said elongated tool body said arms configured to allow stretching of said ligation band over said said arms.
